(19) United States
(12) Patent Application Publication
(10) Pub. No.: US 2005/0036976 A1
(43) Pub. Date: Feb. 17, 2005

(54) Topical skin care composition

(76) Inventors: Joel Rubin, Smithtown, NY (US); Nita Patel, Los Angeles, CA (US)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 10/914,736

(22) Filed: Aug. 9, 2004

Related U.S. Application Data
(60) Provisional application No. 60/494,528, filed on Aug. 12, 2003.

(51) Int. Cl. A61K 7/06; A61K 38/43; A61K 35/72

(52) U.S. Cl. 424/74; 424/94.1; 424/195.16

(57) ABSTRACT

The present invention is directed to a topical skin care composition, comprising: (1) yeast/coenzyme Q₁₀ complex; and (2) a pharmaceutically acceptable topical carrier. The invention is also directed to methods of treating aged skin, and a product sold to treat aged skin.

### CROSS-REFERENCE TO RELATED APPLICATIONS

This application claims the benefit of U.S. Provisional Application Ser. No. 60/494,528 filed Aug. 12, 2003, which is incorporated by reference in its entirety.

### BACKGROUND OF THE INVENTION

1. Field of the Invention

The present invention is directed to topical skin care composition, and more particularly to topical skin care composition that include yeast cells in combination with an antioxidant such as coenzyme Q₁₀.

2. Brief Description of the Related Art

The processes of aging and photoaging are generally associated with an increase in cellular oxidation due to increased presence of damaging free radicals. This phenomenon may be in part due to a decline in the levels of various antioxidants such as coenzyme Q₁₀ (also known as ubiquinone or CoQ₁₀). In one scenario, UVA and UVB exposure can result in formation of free radicals within the skin, and due to low levels of antioxidants from aging or photoaging, such free radicals are not completely removed or scavenged by natural cellular antioxidants.

Coenzyme Q₁₀ is indispensable for ATP production in human metabolism. It is also an important antioxidant in human oxidation defense systems. Coenzyme Q₁₀ is a lipophilic molecule, which means it is easily dissolved in fat, but not in water. Since the skin comprises a large proportion of water, bioavailability of coenzyme Q₁₀ is limited in skin.

Various topical formulations containing coenzyme Q₁₀ have been proposed to combat cellular oxidation. For example, St. Ives Coenzyme Q₁₀ Eye Cream contains coenzyme Q₁₀ and so-called botanicals, and is advertised to reduce the appearance of fine lines and wrinkles around the eyes. A similar product, sold as Nivea Visage Coenzyme Q₁₀ Facial Cream, is advertised for wrinkle control.

Topical formulations containing yeast or yeast extracts have also been proposed for various uses including antiaging.

For example, U.S. Pat. No. 4,540,571 to Schimanski discloses a cosmetic composition for skin care and skin regeneration that includes a yeast extract.

U.S. Pat. Nos. 4,942,031 and 5,023,090 to Levin disclose compositions for treating skin conditions which comprises live yeast cell derivatives and medicinal agents such as antiwrinkling, antibiotic, anticancer, or antifungal agents.

U.S. Pat. No. 5,019,391 to Bunte et al. discloses a skin care composition comprising yeast lysate from brewer's yeast useful for protein biosynthesis in the skin.

U.S. Pat. No. 5,204,105 to Mausner discloses a treatment for the skin under the eyes comprising yeast extracts and other natural ingredients such as vitamin E.

U.S. Pat. No. 5,643,587 to Scancarella et al. and U.S. Pat. No. 5,676,956 to Duffy, et al. disclose topical treatments for skin comprising yeast extracts, magnesium ascorbyl phosphate, vitamin E, and vitamin A.

Although various topical ingredients such as Vitamin E, Vitamin C, and Coenzyme Q₁₀ have been shown to be beneficial for quenching free radicals, their individual bioavailability within the skin has been limited due in part to the anatomy and physiology of the skin itself. Accordingly, what is needed in the art is a topical skin formulation that increases bioavailability of nutrients that are effective to counteract the processes of aging and photoaging. The present invention is believed to be an answer to that need.

### SUMMARY OF THE INVENTION

In one aspect, the present invention is directed to a topical skin care composition, comprising: (1) yeast/coenzyme Q₁₀ complex; and (2) a pharmaceutically acceptable topical carrier.

In another aspect, the present invention is directed to a method of treating aged skin, comprising the step of administering to a patient an effective amount of a topical skin care composition, comprising: (1) yeast/coenzyme Q₁₀ complex; and (2) a pharmaceutically acceptable topical carrier.

In another aspect, the present invention is directed to an article of manufacture comprising packaging material and a topical skin care composition contained within the packaging material, wherein the topical skin care composition is therapeutically effective for treating aged or photoaged skin, and wherein the packaging material comprises a label which indicates that the topical skin care composition can be used for treating aged or photoaged skin, and wherein the topical skin care composition comprises (1) yeast/coenzyme Q₁₀ complex; and (2) a pharmaceutically acceptable topical carrier.

These and other aspects will become apparent upon reading the following detailed description of the invention.

### DETAILED DESCRIPTION OF THE INVENTION

It has now been surprisingly found that a topical composition comprising a yeast/coenzyme Q₁₀ complex in a carrier is effective in treating aged or photoaged skin that has been subjected to oxidation. When applied to the skin, the composition of the invention shows dramatic results in the improvement and disappearance of fine lines and wrinkles and the overall appearance of the skin. While not wishing to be bound by any particular theory, it is believed that the yeast cells used in the composition of the invention have membranes with a higher proportion of coenzyme Q₁₀ relative to naturally occurring amounts, and that this higher proportion of coenzyme Q₁₀ increases the bioavailability of nutrients to the skin, including antioxidants such as coenzyme Q₁₀ polyphenols, glutathiones, beta glucans and isoflavones, that are effective to counteract the processes of oxidation, aging, and photoaging. In addition, it is believed that the higher proportion of coenzyme Q₁₀ in the yeast membranes allows cellular and/or topical compounds that treat aged or photoaged skin to work synergistically to provide a more effective result.

As indicated above, the present invention is directed to a topical skin care composition, comprising: (1) a yeast/coenzyme Q₁₀ complex; and (2) a pharmaceutically acceptable topical carrier. Each of these components is discussed in more detail below.

The yeast/coenzyme Q₁₀ complex component of the invention is a brownish powder, and is the fermentation product of yeast, such as Saccharomyces cerevisiae, fermented in the presence of coenzyme Q₁₀. The yeast species Saccharomyces cerevisiae is particularly useful because it has a unique transportation enzyme superfamily, known as the ABC protein complex, which can transport almost any biological substance regardless of its physical properties. This flexibility gives this yeast an optimum interactive ability with materials in the fermentation medium. It is believed that during fermentation, coenzyme Q₁₀ is transported into the lipophilic portion of the yeast membranes by the ABC protein complex, and remains there due to the lipophilic nature of the coenzyme Q₁₀ molecule. Thus, as defined herein, the phrase “yeast/coenzyme Q₁₀ complex” refers to yeast cells having membranes with increased levels of coenzyme Q₁₀ relative to naturally occurring yeast cells.

Preferably, the yeast/coenzyme Q₁₀ complex component of the invention contains coenzyme Q₁₀ at a minimum concentration of 7.5% by weight of fermented cells. A useful yeast/coenzyme Q₁₀ complex is available from Bio-Foods LTD (Pine Brook, N.J.), and is sold under the trade name “BIO-TRANSFORMED COQ₁₀ YEAST”.

In the composition of the invention, the yeast/coenzyme Q₁₀ complex component preferably comprises from about 0.1 to about 5 wt %, based on the total weight of the composition. A particularly useful amount of yeast/coenzyme Q₁₀ complex component is 2 wt %. Using this amount, the final concentration of coenzyme Q₁₀ is determined to be approximately 0.150% by weight.

The composition of the invention also includes a pharmaceutically acceptable topical carrier. The art is replete with pharmaceutically acceptable topical carriers that may be used in the composition of the invention. In general, however, a desirable topical carrier will feel pleasant on the skin, and maintain the integrity and activity of the yeast/coenzyme Q₁₀ complex. Examples of useful pharmaceutically acceptable topical carriers include, but are not limited to, dimethicone having a spec range of 350 cps; shea butter, or known equivalents such as cocoa butter and the like; hydrogenated castor oil; capric/caprylic triglyceride; ceresine wax, or equivalents, such as ozokerite, micro crystalline wax, and the like; petrolatum; cetyl dimethicone copolyol; BIS-diglyceryl polyacyladipate-2; behenoyl stearic acid; dibehenoyl fumarate; polyethylene glycol-30 dipolyhydroxystearate; and combinations thereof.

Preferably, the amount of carrier in the topical skin care composition of the invention ranges from about 15 to about 75 wt %, more preferably from about 25 to about 65 wt %, and most preferably from about 30 to about 50 wt %, all percentages based on the total weight of the composition. The appropriate amount of carrier may be easily determined by one skilled in the art.

Water may also be included in the topical skin care composition of the invention. The water component generally represents the balance between the other ingredients and 100%. Thus, the appropriate amount of water is added to the composition of the invention to raise the total ingredient levels to 100% by weight.

The composition may also include additional ingredients, such as emulsifiers, preservatives, chelating agents, fragrances, natural aromas, moisturizers, and the like, that are all well known in the skin care art. In one embodiment, the topical skin care composition of the invention includes an emulsifier such as sorbitan stearate, sodium chloride, sodium borate. As known in the art, combinations of two, three, four, or more of these emulsifiers may also be implemented in the composition of the invention, for example, sodium chloride and cetyl dimethicone copolyol; or behenoyl stearic acid and sodium borate.

Preferably, the amount of emulsifier in the topical skin care composition of the invention ranges from about 0.5 to about 7 wt %, more preferably, from about 4 to about 7 wt %, and most preferably from about 5 to about 7 wt %, all weight percents being based on the total weight of the composition.

Useful preservatives that may be included in the topical skin care composition of the invention include propylparaben, methylparaben, diazolidinylurea, “GLYDANT PLUS” (generically know as DMDM hydantoin and iodobutylcarbamate, and available from Lonza. Inc., Mapleton, Ill.) phenoxyethanol, chlorphenisin, and the like. As known in the art, combinations of two, three, four, or more of these preservatives may also be implemented in the composition of the invention. Useful amounts of preservatives in the composition of the invention generally from about 0.1 to about 5 wt %, and more preferably from about 0.1 to about 1 wt %, based on the total weight of the composition.

Further additional ingredients may also be included in the topical skin care composition of the invention. Such additional ingredients include pentylene glycol (a preservative extender), chelating agents, fragrances, moisturizers, humectants, fragrances, natural aromas, as well as combinations of these. Useful natural aromas include ginger oil, grapefruit oil, chamomile oil, orange oil, rosemary oil, etc. Useful amounts of these additional ingredients generally range from about 0.1 to about 5 wt %, and more preferably from about 0.1 to 1.5 wt %, based on the total weight of the composition.

In preferred embodiments, the topical skin care composition of the invention has the ingredients and preferred ranges shown in Table 1:

TABLE 1

| | | More | Most |
|---|---|---|---|
| | Preferred | Preferred | Preferred |
| | Range | Range | Range |
| Ingredient | (wt %) | (wt %) | (wt %) |
| Dimethicone | 3-10 | 4-9 | 5-7 |
| Shea Butter | 1-7 | 2-6 | 2-4 |
| Propylparaben | 0.1 | 0.1 | 0.1 |
| Hydrogenated Castor Oil | 0.5-2.5 | 0.75-2.0 | 1.0-1.5 |
| Sorbitan Stearate | 0.2-1.0 | 0.4-0.8 | 0.5-0.7 |
| Capric/Caprylic Triglyceride | 5.0-15.0 | 7.5-12.5 | 8.0-10.0 |
| Ceresine Wax | 0.5-2.5 | 0.75-2.0 | 1.0-1.5 |
| Petrolatum | 5.0-15.0 | 7.5-12.5 | 8.0-10.0 |
| Cetyl Dimethicone Copolyol | 1.0-5.0 | 1.5-3.0 | 1.5-2.0 |
| BIS-Diglyceryl Polyacyladipate-2 | 2.0-8.0 | 3.0-7.0 | 4.0-6.0 |
| Behenoyl Stearic Acid | 1.0-4.0 | 2.0-3.5 | 2.0-3.0 |
| Dibehenyl Fumarate | 0.1-1.0 | 0.2-0.8 | 0.2-0.5 |
| PEG-30 Dipolyhydroxystearate | 0.3-1.5 | 0.5-1.25 | 0.7-1.0 |
| Water (Purified) | balance | balance | balance |
| Yeast/Coenzyme Q₁₀ Complex | 2.0 | 2.0 | 2.0 |
| Tetrasodium EDTA | 0.02-0.20 | 0.04-0.16 | 0.04-0.10 |
| Pentylene Glycol | 2.5 | 2.5 | 2.5 |
| Methylparaben | 0.25 | 0.25 | 0.25 |
| Sodium Borate | 0.4-1.6 | 0.8-1.4 | 0.8-1.2 |
| Sodium Chloride | 0.2-1.0 | 0.3-0.9 | 0.4-0.8 |
| Diazolidinyl Urea | 0.3 | 0.3 | 0.3 |
| Glydant Plus | 0.05 | 0.05 | 0.05 |
| Ginger Oil | 0.1-0.5 | 0.2-0.4 | 0.2-0.3 |
| Florida Grapefruit Oil | 0.1-0.4 | 0.1-0.3 | 0.1-0.2 |
| Moroccan Chamomile Oil | 0.1-0.6 | 0.2-0.5 | 0.3-0.4 |
| Bitter Orange Oil | 0.1-0.5 | 0.1-0.4 | 0.1-0.3 |
| Spanish Rosemary Oil | 0.1-0.6 | 0.2-0.5 | 0.3-0.4 |

To prepare the composition of the invention, the components of the carrier portion are first combined into a single carrier mixture. Separately, a yeast mixture and a preservative mixture are individually prepared, combined, and added to the carrier mixture, along with any optional ingredients. The composition of the invention may be made in small batches using a standard blender, or in large mixing vats. The temperatures of the mixtures and the formulation process may be maintained by conventional temperature apparatus, such as heaters. Details of the preparation process is described in more detail in the Examples below.

The composition may be applied to any part of the skin that is suffering from aging or photoaging (oxidation). In use, approximately ½ gram of the composition of the invention would be applied for full coverage of the face. More or less than ½ gram may be used depending on the severity of the conditions and area of skin sought to be treated. The composition of the invention may be applied from 1 to 5 times per day, and preferably once or twice per day. In certain cases, it may be desirable to apply the composition of the invention at night before bedtime.

When applied to aged, photoaged, or otherwise oxidized skin, the composition of the invention provides significant improvement in skin texture, elasticity/tone, clarity, dryness, roughness, fine lines and wrinkles and overall appearance.

As indicated above, the invention also includes an article of manufacture comprising packaging material and the topical skin care composition of the invention contained within the packaging material, preferably in a tube or bottle dispenser. Such an article of manufacture may be sold commercially through conventional channels such as a drug store, salon, or department store, or, in the alternative, through other channels such as telemarketing ads or from a dermatologist's office.

### EXAMPLES

The following examples are intended to illustrate, but in no way limit the scope of the present invention. All parts and percentages are by weight and all temperatures are in degrees Celsius unless explicitly stated otherwise.

### Example 1

### Preparation of a Topical Antioxidant Composition

A topical antioxidant composition was prepared as follows. A first batch of ingredients (“Phase A”) shown in Table 2 was heated to 85° C. and mixed until uniform.

TABLE 2
Phase A

| Ingredient | Final Wt % |
|---|---|
| Dimethicone | 5.00 |
| Cetyl PEG/PPG 10/1 Dimethicone | 1.75 |
| Butyrospermum Parkii (Shea Butter) | 2.25 |
| Bis-Diglyceryl Polyacyladipate-2 | 5.00 |
| Propylparaben | 0.10 |
| Behenoyl Stearic Acid | 2.25 |
| Hydrogenated Castor Oil | 1.00 |
| Sorbitan Stearate | 0.50 |
| Dibehenyl Fumarate | 0.25 |
| PEG-30 Dipolyhydroxystearate | 1.00 |
| Caprylic/Capric Triglyceride | 8.00 |
| Ceresine Wax | 1.00 |
| Petrolatum | 10.00 |

Separately, a second batch of ingredients (“Phase B”) shown in Table 3 was heated to 35° C. and mixed until uniform.

TABLE 3
Phase B

| Ingredient | Final Wt % |
|---|---|
| Water (Purified) | 54.20 |
| Yeast/Coenzyme Q₁₀ Complex | 2.0 |
| (40%) Tetrasodium EDTA | 0.1 |
| Sodium Chloride | 0.4 |
| Sodium Borate | 0.9 |
| Diazolidinyl Urea | 0.3 |

Separately, a third batch of ingredients (“Phase C”) shown in Table 4 was heated to 40° C. and mixed until all the ingredients were solubilized.

TABLE 4
Phase C

| Ingredient | Final Wt % |
|---|---|
| Pentylene Glycol | 2.50 |
| Methylparaben | 0.25 |
| “Glydant Plus” (DMDM Hydantoin and | 0.05 |
| Iodopropynyl Butylcarbamate | |

Phase C is then added to Phase B, and the two phases are mixed until homogeneous. The combined Phases B and C are then slowly added to Phase A while homomixing at approximately 2000 rpm. The homomixing continues for about five (5) minutes after last portions of combined Phases B and C are added. Following this mix, the homomixer is turned off and the mixture is allowed to cool to approximately 40° C. while sweepmixing (also known as planetary mixing).

Separately, a fourth batch of ingredients (“Phase D”) shown in Table 5 was mixed until uniform.

TABLE 5
Phase D

| Ingredient | Final Wt % |
|---|---|
| Ginger Oil | 0.25 |
| Grapefruit Oil | 0.15 |
| Chamomile Oil | 0.30 |
| Bitter Orange Oil | 0.20 |
| Spanish Rosemary Oil | 0.30 |

Phase D is then added to the combined phases A, B, and C and mixed at 30° C. until homogeneous.

### Example 2

### Topical Antioxidant Composition Study

The benefits of the topical antioxidant composition made in Example 1 was evaluated on human skin. Briefly, 20 patients were enrolled in a double blinded placebo-controlled study. Patients ages 25 to 80 years old were enrolled; 17 females, 3 males. 19 patients completed the study. Patients were instructed to use a gentle cleanser and sunblock five days prior to the study to washout any effects of any previous products used.

The topical antioxidant composition described in Example 1 was used for evaluation. As a control (placebo), the same composition without the 2% yeast/coenzyme Q₁₀ was employed. The patients were instructed to use the gentle cleanser and sunblock on the entire face in the morning. In the evening, the patients used the gentle cleanser. The patients were then randomly assigned to use Composition #1 (active cream) on half the face and Composition #2 (placebo) on the opposite side. The creams were blinded for both the patient and physician. The patients were evaluated by survey, ultrasound, and digital photography at 7, 14 and 30 days.

Survey Results

Following 7, 14, and 30 days of treatment, patients were given a survey asking them to rate several factors contributing to overall skin appearance, including skin texture, skin elasticity and tone, skin redness or blotchiness, skin pigmentation, skin clarity, oiliness, dryness, roughness, improvement and disappearance of fine lines and wrinkles, and pores. Each factor was assigned a numerical value as follows: 1=no improvement; 2=slight improvement; 3=moderate improvement; 4=excellent improvement. Control negative questions were included in the survey to assess the accuracy of the positive responses. The numerical results for each factor are added for the appropriate time frame. The results are shown in Table 6.

TABLE 6
Survey Results

| | SLIGHT TO | | | MODERATE TO | | |
|---|---|---|---|---|---|---|
| | EXCELLENT | | | EXCELLENT | | |
| Attribute | Day 7 | Day 14 | Day 30 | Day 7 | Day 14 | Day 30 |
| Skin Texture | | | | | | |
| Comp. 1 | 58 | 63 | 74 | 32 | 37 | 58 |
| (Active) | | | | | | |
| Comp. 2 | 47 | 47 | 63 | 26 | 21 | 37 |
| (Placebo) | | | | | | |
| Elasticity/Tone | | | | | | |
| Comp. 1 | 32 | 53 | 63 | 26 | 32 | 47 |
| (Active) | | | | | | |
| Comp. 2 | 26 | 32 | 58 | 16 | 16 | 26 |
| (Placebo) | | | | | | |
| Redness/ | | | | | | |
| Blotchiness | | | | | | |
| Comp. 1 | 21 | 26 | 42 | 16 | 16 | 26 |
| (Active) | | | | | | |
| Comp. 2 | 11 | 16 | 37 | 0 | 5 | 11 |
| (Placebo) | | | | | | |
| Pigmentation | | | | | | |
| Comp. 1 | 11 | 11 | 37 | 0 | 0 | 16 |
| (Active) | | | | | | |
| Comp. 2 | 11 | 5 | 26 | 0 | 0 | 0 |
| (Placebo) | | | | | | |
| Clarity | | | | | | |
| Comp. 1 | 53 | 42 | 58 | 32 | 32 | 47 |
| (Active) | | | | | | |
| Comp. 2 | 42 | 37 | 58 | 16 | 21 | 21 |
| (Placebo) | | | | | | |
| Oiliness | | | | | | |
| Comp. 1 | 26 | 42 | 37 | 5 | 26 | 11 |
| (Active) | | | | | | |
| Comp. 2 | 32 | 21 | 32 | 5 | 21 | 5 |
| (Placebo) | | | | | | |
| Dryness | | | | | | |
| Comp. 1 | 68 | 53 | 58 | 42 | 32 | 58 |
| (Active) | | | | | | |
| Comp. 2 | 68 | 58 | 53 | 32 | 26 | 42 |
| (Placebo) | | | | | | |
| Roughness | | | | | | |
| Comp. 1 | 47 | 53 | 58 | 16 | 21 | 47 |
| (Active) | | | | | | |
| Comp. 2 | 47 | 47 | 53 | 21 | 5 | 26 |
| (Placebo) | | | | | | |
| Wrinkles/ | | | | | | |
| Fine Lines | | | | | | |
| Comp. 1 | 32 | 53 | 68 | 5 | 26 | 37 |
| (Active) | | | | | | |
| Comp. 2 | 21 | 21 | 53 | 5 | 5 | 21 |
| (Placebo) | | | | | | |
| Pores | | | | | | |
| Comp. 1 | 32 | 37 | 42 | 21 | 21 | 32 |
| (Active) | | | | | | |
| Comp. 2 | 26 | 21 | 37 | 16 | 21 | 16 |
| (Placebo) | | | | | | |
| Overall | | | | | | |
| Appearance | | | | | | |
| Comp. 1 | 53 | 63 | 68 | 32 | 42 | 58 |
| (Active) | | | | | | |
| Comp. 2 | 53 | 53 | 68 | 32 | 32 | 63 |
| (Placebo) | | | | | | |

As shown in Table 6, the active cream containing yeast complex (Composition 1) exhibited significant improvement in skin texture, elasticity/tone, clarity, dryness, roughness, fine lines and wrinkles and overall appearance in all cases Placebo showed improvement in dryness and overall appearance, likely attributed to the moisturizing effects of the vehicle.

Digital Photography

Following 7, 14, and 30 days of treatment, patients were evaluated by digital photography. Although all patient showed some kind of improvement in appearance, following examination of the digital photographs, seven patients showed dramatic improvement in fine lines and wrinkles.

Ultrasound

Following 7, 14, and 30 days of treatment, patients were evaluated by ultrasound which provides a measure of collagen density (density of the dermis). Briefly, a 20 MHz ultrasound instrument (DUB Plus instrument from tpm taberna pro medicum/USA, GWB International, Ltd., Marshfield Hills, Mass.) was used to determine collagen density before and after treatment. In the present analysis, the system parameters were set to a scanning frequency of approximately 20 MHz, with a resolution of approximately 72 μm, and a scanning distance of approximately 12.8 mm. The patient with most dramatic results showed significant changes in collagen density (production) with ultrasound.

Discussion

The data was evaluated in a blind manner as Composition 1 (active cream) versus Composition 2 (placebo). Surprisingly, many patients identified Composition 1 (active cream) as beneficial as early as 7 days. Over 30% of patients reported moderate to excellent improvement with Composition 1 (active cream) in skin texture, clarity, dryness and overall appearance in 7 days. Following 30 days use, over 35% patients reported moderate to excellent improvement with Composition 1 (active cream) in skin texture, elasticity/tone, clarity, dryness, roughness, fine lines and wrinkles and overall appearance. Composition 2 (placebo) reported at least a 10% less effect compared to Composition 1 (active cream) except for overall appearance.

No significant improvement was reported in Composition 1 (active cream) or Composition 2 (placebo) for control questions which were predicted to have no effect. This included no effect for redness, blotchiness, pigmentation, oiliness and pores. A greater than 30% improvement in dryness and overall improvement was reported by Composition 2 (placebo).

The results suggest that there was a gradual increase in the percent of patients reporting both slight to excellent and moderate to excellent improvement with the use of the active cream over the course of 30 days. 68% of patients reported slight to excellent improvement in fine lines and wrinkles. Digital photography documented drastic improvement in 33 % of the patients. Ultrasound changes were also seen in correlation with the most dramatic improvement in wrinkles around the mouth.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entireties.

1. A topical skin care composition, comprising:
(1) yeast/coenzyme Q₁₀ complex; and
(2) a pharmaceutically acceptable topical carrier.

2. The topical skin care composition of claim 1, wherein the amount of said yeast/coenzyme Q₁₀ complex ranges from about 0.1 to 5 wt %, based on the total weight of said composition.

3. The topical skin care composition of claim 1, wherein said pharmaceutically acceptable topical carrier is selected from the group consisting of dimethicone having a spec range of 350 cps, shea butter, cocoa butter, hydrogenated castor oil, capric/caprylic triglyceride, ceresine wax, ozokerite, microcrystalline wax, petrolatum, cetyl dimethicone copolyol, BIS-diglyceryl polyacyladipate-2, behenoyl stearic acid, dibehenoyl fumarate, polyethylene glycol-30 dipolyhydroxystearate, and combinations thereof.

4. The topical skin care composition of claim 1, wherein said pharmaceutically acceptable topical carrier further comprises an emulsifier.

5. The topical skin care composition of claim 4, wherein said emulsifier is selected from the group consisting of sorbitan stearate, sodium chloride, sodium borate, sodium chloride and cetyl dimethicone copolyol; behenoyl stearic acid and sodium borate, and combinations thereof.

6. The topical skin care composition of claim 1, further comprising a preservative.

7. The topical skin care composition of claim 6, wherein said preservative is selected from the group consisting of propylparaben, methylparaben, diazolidinylurea, DMDM hydantoin and iodobutylcarbamate (“GLYDANT PLUS”), phenoxyethanol, chlorphenisin, and combinations thereof.

8. The topical skin care composition of claim 1, further comprising additional ingredients selected from the group consisting of pentylene glycol, chelating agents, fragrances, moisturizers, humectants, natural aromas, and combinations thereof.

9. The topical skin care composition of claim 8, wherein said natural aromas are selected from the group consisting of ginger oil, grapefruit oil, chamomile oil, orange oil, rosemary oil, and combinations thereof, and wherein the amount of said natural aromas ranges from about 0.1 to about 5 wt %, based on the total weight of said composition.

10. The topical skin care composition of claim 1, wherein the amount of said carrier in said composition ranges from about 15 to about 75 wt %, based on the total weight of said composition.

11. The topical skin care composition of claim 10, wherein the amount of said carrier in said composition ranges from about 25 to about 65 wt %, based on the total weight of said composition.

12. The topical skin care composition of claim 11, wherein the amount of said carrier in said composition ranges from about 30 to about 50 wt %, based on the total weight of said composition.

13. The topical skin care composition of claim 6, wherein the amount of said preservative in said composition ranges from about 0.1 to about 5 wt %, based on the total weight of said composition.

14. The topical skin care composition of claim 4, wherein the amount of said emulsifier in said composition ranges from about 0.5 to about 7 wt %, based on the total weight of said composition.

15. The topical skin care composition of claim 14, wherein the amount of said emulsifier in said composition ranges from about 4 to about 7 wt %, based on the total weight of said composition.

16. The topical skin care composition of claim 15, wherein the amount of said emulsifier in said composition ranges from about 5 to about 7 wt %, based on the total weight of said composition.

17. The topical skin care composition of claim 1, further comprising water.

18. A method of treating aged skin, comprising the step of administering to a patient an effective amount of a topical skin care composition, comprising:
(1) yeast/coenzyme Q₁₀ complex; and
(2) a pharmaceutically acceptable topical carrier.

19. The method of claim 18, wherein said topical skin care composition, comprises about 2 wt of said yeast/coenzyme Q₁₀ complex, based on the total weight of said composition.

20. The method of claim 18, wherein said effective amount of said topical skin care composition comprises approximately ½ gram.

21. The method of claim 18, wherein said topical skin care composition is applied to the skin from 1 to 5 times per day.

22. An article of manufacture comprising packaging material and a topical skin care composition contained within said packaging material, wherein said topical skin care composition is therapeutically effective for treating aged or photoaged skin, and wherein said packaging material comprises a label which indicates that said topical skin care composition can be used for treating aged or photoaged skin, and wherein said topical skin care composition comprises (1) yeast/coenzyme Q₁₀ complex; and (2) a pharmaceutically acceptable topical carrier.
